(19) United States
(12) Patent Application Publication
(10) Pub. No.: US 2011/0004076 A1
(43) Pub. Date: Jan. 6, 2011

(54) SYSTEM AND METHOD FOR COMMUNICATING WITH AN IMPLANT

(75) Inventors: Sied W. Janna, Memphis, TN (US); Darren James Wilson, Yorkshire (GB); Andrew Jon Fell, Cambridge (GB); David Roger Tegerdine, Royston (GB); Abi Claire Graham, Cambridge (GB); Stephen Russell Taylor, Cambridge (GB)

Correspondence Address: Smith & Nephew, Inc.;Diana Houston, 1450 Brooks Road, Memphis, TN, 38116, US

(73) Assignee: SMITH & NEPHEW, INC., Memphis, TN (US)

(21) Appl. No.: 12/865,657

(22) PCT Filed: Jan. 30, 2009
(86) PCT No.: PCT/US2009/032540

§ 371 Date: Jul. 30, 2010

Related U.S. Application Data
(60) Provisional application No. 61/025,362, filed on Feb. 1, 2008.
(60) Provisional application No. 61/044,295, filed on Apr. 11, 2008.

(51) Int. Cl. A61B 5/07; H04B 5/00

(52) U.S. Cl. 600/302; 455/41.1

(57) ABSTRACT

A system and method for communicating with a medical implant is disclosed. The system (10,210,310,410) includes on-board electronics, a signal generator (15,215), an amplifier (16,216), a coil (14,214), a receiver (22,222), and a processor (20,220). The on-board electronics (100, 110) include a power harvester, a sensor, a microprocessor, and a data transmitter. The signal generator (15,215) generates a first signal, the amplifier (16,216) amplifies the first signal, the coil (14,214) transmits the amplified signal, the power harvester receives the first signal and transmits a data packet (18,218) containing data, the receiver (22,222) receives the data packet (18,218), and the processor (20,220) either processes the data or sends the data to a data storage device.

### CROSS-REFERENCE TO RELATED APPLICATIONS

This application claims the benefit of U.S. Provisional Application No. 61/025,362, filed on Feb. 1, 2008 and U.S. Provisional Application No. 61/044,295, filed on Apr. 11, 2008. The disclosure of each prior application is incorporated by reference in its entirety.

### BACKGROUND OF THE INVENTION

1. Field of the Invention

The present invention relates generally to orthopaedic implants and more particularly to orthopaedic implants that incorporate a portion of a radio telemetry system.

2. Related Art

Trauma products, such as intramedullary (IM) nails, pins, rods, screws, plates and staples, have been used for many years in the field of orthopaedics for the repair of broken bones. These devices function well in most instances, and fracture healing occurs more predictably than if no implant is used. In some instances, however, improper installation, implant failure, infection or other conditions, such as patient non-compliance with prescribed post-operative treatment, may contribute to compromised healing of the fracture, as well as increased risk to the health of the patient.

Health care professionals currently use non-invasive methods, such as x-rays, to examine fracture healing progress and assess condition of implanted devices. However, x-rays may be inadequate for accurate diagnoses. They are costly, and repeated x-rays may be detrimental to the patient's and health care workers' health. In some cases, non-unions of fractures may go clinically undetected until implant failure. Moreover, x-rays may not be used to adequately diagnose soft tissue conditions or stress on the implant. In some instances, invasive procedures are required to diagnose implant failure early enough that appropriate remedial measures may be implemented.

The trauma fixation implants currently available on the market are passive devices because their primary function is to support the patient's weight with an appropriate amount of stability whilst the surrounding fractured bone heals. Current methods of assessing the healing process, for example using radiography or patient testimonial do not provide physicians with sufficient information to adequately assess the progress of healing, particularly in the early stages of healing. X-ray images only show callus geometry and cannot access the mechanical properties of the consolidating bone. Therefore, it is impossible to quantify the load sharing between implant and bone during fracture healing from standard radiographs, CT, or MRI scans. Unfortunately, there is no in vivo data available quantifying the skeletal loads encountered during fracture healing as well as during different patient and physiotherapy activities. The clinician could use this information to counsel the patient on life-style changes or to prescribe therapeutic treatments if available. Continuous and accurate information from the implant during rehabilitation would help to optimize postoperative protocols for proper fracture healing and implant protection and add significant value in trauma therapy. Furthermore, improvements in security, geometry, and speed of fracture healing will lead to significant economic and social benefits. Therefore, an opportunity exists to augment the primary function of trauma implants to enhance the information available to clinicians.

Patient wellness before and after an intervention is paramount. Knowledge of the patient's condition can help the caregiver decide what form of treatment may be necessary given that the patient and caregiver are able to interact in an immediate fashion when necessary. Many times the caregiver does not know the status of a would-be or existing patient and, therefore, may only be able to provide information or incite after it was necessary. If given information earlier, the caregiver can act earlier. Further, the earlier information potentially allows a device to autonomously resolve issues or remotely perform the treatment based on a series of inputs.

Surgeons have historically found it difficult to assess the patient's bone healing status during follow up clinic visits. It would be beneficial if there was a device that allowed the health care provider and patient to monitor the healing cascade. Moreover, it would be beneficial if such a device could assist in developing custom care therapies and/or rehabilitation.

Wireless technology in devices such as pagers and hand-held instruments has long been exploited by the healthcare sector. However, skepticism of the risks associated with wireless power and communication systems has prevented widespread adoption, particularly in orthopaedic applications. Now, significant advances in microelectronics and performance have eroded many of these perceived risks to the point that wireless technology is a proven contender for high integrity medical systems. Today's medical devices face an increasingly demanding and competitive market. As performance targets within the sector continue to rise, new ways of increasing efficiency, productivity and usability are sought. Wireless technology allows for two-way communication or telemetry between implantable electronic devices and an external reader device and provides tangible and recognized benefits for medical products and is a key technology that few manufacturers are ignoring.

Currently, Radio Frequency (RF) telemetry and inductive coupling systems are the most commonly used methods for transmitting power and electronic data between the implant and the companion reader Implantable telemetric medical devices typically utilize radio-frequency energy to enable two way communications between the implant and an external reader system. Although data transmission ranges in excess of 30 m have been observed previously, energy coupling ranges are typically reduced to a couple of inches using wireless magnetic induction making these implants unsuitable for commercial application. Power coupling issues can be minimized using a self-contained lithium battery, which are typically used in active implantable devices such as pacemakers, insulin pumps, neurostimulators and cochlea implants. However, a re-implantation procedure must be performed when the battery is exhausted, and a patient obviously would prefer not to undergo such a procedure if possible.

Some telemetric systems include electronics and/or an antenna. In general, these items must be hermetically sealed to a high standard because many electronic components contain toxic compounds, some electronic components need to be protected from moisture, and ferrite components, such as the antenna, may be corroded by bodily fluids, potentially leading to local toxicity issues. Many polymers are sufficiently biocompatible for long-term implantation but are not sufficiently impermeable and cannot be used as encapsulants or sealing agents. In general, metals, glasses, and some ceramics are impermeable over long timescales and may be better suited for use in encapsulating implant components in some instances.

Additionally, surgeons have found it difficult to manage patient information. It would be beneficial if there was available a storage device that stored patient information, such as entire medical history files, fracture specifics, surgery performed, X-ray images, implant information, including manufacturer, size, material, etc. Further, it would be beneficial if such storage device could store comments/notes from a health care provider regarding patient check-ups and treatments given.

### SUMMARY OF THE INVENTION

According to some aspects of the present invention there may be provided a system for communicating patient information. The system may include a medical implant, the medical implant has a first cavity and a second cavity, the first and second cavity connected by one or more apertures, the first cavity is adapted to receive on-board electronics, the on-board electronics comprising at least one sensor, a microprocessor, and a data transmitter, and the second cavity is adapted to receive an implant antenna; a signal generator adapted to generate a first signal; an amplifier electrically connected to the signal generator; at least one coil electrically connected to the amplifier; a receiver adapted to receive a data packet having data from the implant antenna; and a processor connected to the receiver; wherein the signal generator generates the first signal, the amplifier amplifies the first signal, the at least one coil transmits the amplified signal, the implant antenna receives the first signal and transmits a data packet containing data, the receiver receives the data packet, and the processor either processes the data or sends the data to a data storage device.

According to some embodiments, the processor is selected from the group consisting of a desktop computer, a laptop computer, a personal data assistant, a mobile handheld device, and a dedicated device.

According to some embodiments, the receiver may be an antenna with an adapter for connection to the processor.

According to some embodiments, the on-board electronics may include a plurality of sensor assemblies and a multiplexer.

According to some embodiments, the at least one coil may be a transmission coil.

According to some embodiments, there are two coils, and the coils are housed within a paddle.

According to some embodiments, the system further includes a control unit, and wherein the signal generator and the amplifier are housed within the control unit.

According to some embodiments, the system further includes one or more components selected from the group consisting of a feedback indicator, a load scale, a portable storage device, a second processor.

According to some embodiments, the first signal has a frequency of about 125 kHz.

According to some embodiments, the first cavity and the second cavity are orthogonal to one another.

According to some embodiments, the first cavity and the second cavity are diametrically opposed.

According to some embodiments, at least one of the first cavity and the second cavity further includes a cover.

According to some embodiments, the on-board electronics comprise an LC circuit, a bridge rectifier, a storage capacitor, a wake up circuit, a microprocessor, an enable measurement switch, an amplifier, a Wheatstone bridge assembly, and a modulation switch.

According to some embodiments, the microprocessor may include an analog to digital converter.

According to some embodiments, the modulation switch may modulate a load signal. According to some embodiments, the load signal may be modulated at a frequency between 5 kHz and 6 kHz.

The invention includes a system having a telemetric implant. The telemetric implant is capable of receiving power wirelessly from an external reader at a distance using sophisticated digital electronics, on board software, and radio frequency signal filtering. The implant may be equipped with at least one sensor, interface circuitry, micro-controller, wakeup circuit, high powered transistors, printed circuit board, data transmitter and power receive coil with software algorithm, all of which may be embedded in machined cavities located on the implant. The telemetry system may use a coiled ferrite antenna housed and protected inside the metallic body of the implant using a metal encapsulation technique suitable for long term implantation. The use of digital electronics and a high permeable material located inside a metallic cavity compensates for the effect of severely shielding a power coil from the externally applied magnetic power field. The digital electronics enables multiplexing to read multiple sensors. The electronics module does not require the reader to be positioned within a pre-defined “sweet spot” over the implant in order to achieve a stable reading relating to sensed data minimizing the potential to collect erroneous measurements.

Further areas of applicability of the invention will become apparent from the detailed description provided hereinafter. It should be understood that the detailed description and specific examples, while indicating the particular embodiment of the invention, are intended for purposes of illustration only and are not intended to limit the scope of the invention.

### BRIEF DESCRIPTION OF THE DRAWINGS

The accompanying drawings, which are incorporated in and form a part of the specification, illustrate the embodiments of the present invention and together with the written description serve to explain the principles, characteristics, and features of the invention. In the drawings:

FIG. 1 illustrates a first system for communicating with an implant;

FIG. 2 illustrates a block diagram for power harvesting;

FIG. 3 illustrates a block diagram for signal transmission;

FIG. 4 illustrates an exemplary data packet structure;

FIG. 5 illustrates an exemplary receiver circuit board;

FIG. 6 illustrates a flowchart showing the reader steps;

FIG. 7 illustrates an exemplary electrical diagram of the implant electronics;

FIG. 8 illustrates a flowchart showing the steps of sensor measurement;

FIG. 9 illustrates a first embodiment of on-board implant electronics;

FIG. 10 illustrates a second embodiment of on-board implant electronics;

FIGS. 11-14 illustrate one particular embodiment of the orthopaedic implant;

FIG. 15 illustrates a first cavity and a second cavity;

FIGS. 16-23 illustrate assembly of the orthopaedic implant shown in FIGS. 11-14;

FIG. 24 illustrates a second system for communicating with an implant;

FIG. 25 illustrates a coil;

FIG. 26 illustrates a third system for communicating with an implant;

FIG. 27 illustrates a paddle;

FIG. 28 illustrates a wiring diagram of the paddle and the receiver;

FIG. 29 illustrates a fourth system for communicating with an implant;

FIG. 30 is a graph illustrating the received signal of the fourth system;

FIG. 31 illustrates a data storage system; and

FIG. 32 illustrates a health care facility with one or more kiosks.

### DETAILED DESCRIPTION OF THE EMBODIMENTS

The following description of the depicted embodiment(s) is merely exemplary in nature and is in no way intended to limit the invention, its application, or uses.

A “smart implant” is an implant that is able to sense its environment, apply intelligence to determine whether action is required, and possibly act on the sensed information to change something in a controlled, beneficial manner This would ideally occur in a closed feedback loop reducing the chance of coming to an erroneous conclusion when evaluating the sensed data. One attractive application of smart implant technology is to measure loads on an orthopaedic implant. For example, an intramedullary nail subjected to six spacial degrees of freedom, comprised of 3 forces (Axial Force, Fz, Shear Force Fx & Fy) and 3 moments (Mx-bending, My-bending and Mz-torsional) may be measured indirectly by measuring sensor output of a series of strain gauges mounted to the orthopaedic implant using the matrix method.

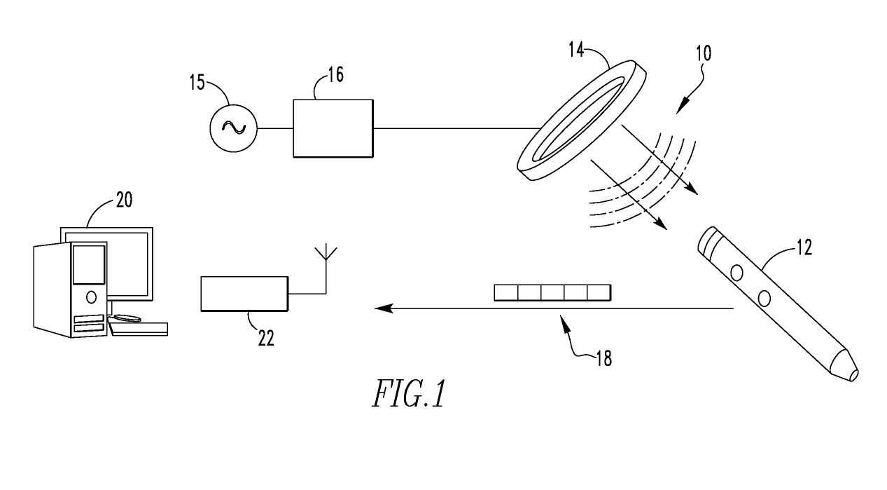

FIG. 1 illustrates a system 10 for communicating with an implant in a first embodiment. The system 10 includes an orthopaedic implant 12, a coil 14, a signal generator 15, an amplifier 16, a data packet 18, a processor 20, and a receiver 22. In the depicted embodiment, the orthopaedic implant is an intramedullary nail but other types of orthopaedic implants may equally be used. As examples, the orthopaedic implant may be an intramedullary nail, a bone plate, a hip prosthetic, or a knee prosthetic. Further, the processor 20 is depicted as a desktop computer in FIG. 1 but other types of computing devices may equally be used. As examples, the processor 20 may be a desktop computer, a laptop computer, a personal data assistant (PDA), mobile handheld device, or a dedicated device. In some embodiments, the processor 20 and the receiver 22 form a single component. In the depicted embodiment, however, the receiver 22 is electrically connected to the processor 20 but is a separate component. As examples, the receiver 22 may be an antenna with an adapter to connect to a computer port or a wireless interface controller (also known as a wireless card) for connection to the processor 20, such as through the use of a PCI bus, mini PCI, PCI Express Mini Card, USB port, or PC Card. As is explained in greater detail below, the signal generator 15 generates a signal, the amplifier 16 amplifies the signal, the coil 14 transmits the amplified signal, the orthopaedic implant 12 receives the signal and transmits a data packet 18 containing data, the receiver 22 receives the data packet, and the processor 20 may either process the data or send the data to a storage device (not shown).

The orthopaedic implant 12 may incorporate one or more power management strategies. Power management strategies may include implanted power sources or inductive power sources. Implanted power sources may be something simple, such as a battery, or something more complex, such as energy scavenging devices. Energy scavenging devices may include motion powered piezoelectric or electromagnetic generators and associated charge storage devices. Inductive power sources include inductive coupling systems and Radio Frequency (RF) electromagnetic fields. The orthopaedic implant 12 may incorporate a storage device (not shown). The storage device may be charged by an inductive/RF coupling or by an internal energy scavenging device. Preferably, the storage device has sufficient capacity to store enough energy at least to perform a single shot measurement and to subsequently process and communicate the result.

In some embodiments, the orthopaedic implant 12 may be inductively powered. FIG. 2 illustrates an exemplary block diagram for harvesting power from the amplified signal. The assembled components, which may form a portion of printed circuit board or a separate assembly, generally is referred to as a power harvester 30. The power harvester 30 includes an antenna 32, a rectifier 34, and a storage device 36. In the depicted embodiment, the storage device 36 is a capacitor but other devices may be used.

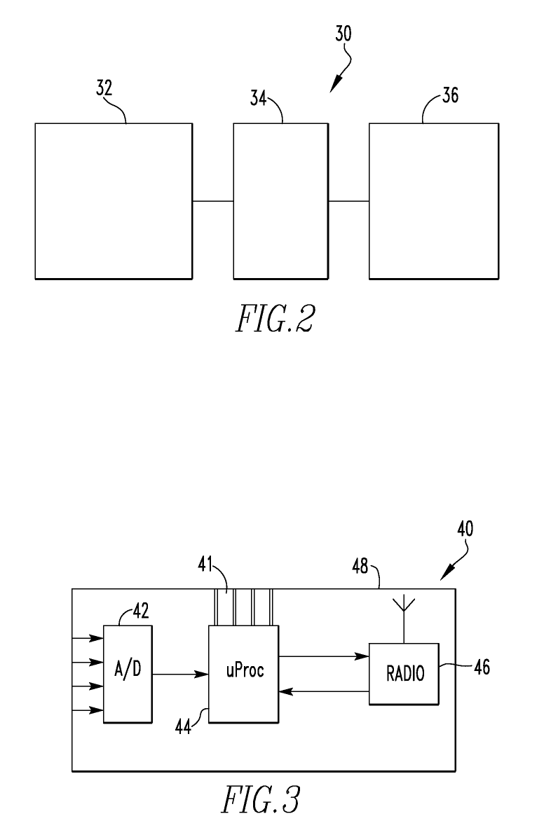

In some embodiments, the orthopaedic implant 12 may include an onboard microchip that converts signals from analog to digital and sends the digital signal via a radio wave. FIG. 3 illustrates an exemplary block diagram of a microchip 40 for signal conversion and signal transmission. The microchip 40 also may be termed a microcontroller. The microchip 40 includes a converter 42, a processor 44, a transmitter 46, and an antenna 48. The converter 42 converts analog signals to digital signals. The processor 44 is electrically connected to the converter 42. In some embodiments, the processor 44 is also connected to an input/output port 41. The transmitter 46 is electrically connected to the processor 44 and to the antenna 48. In some embodiments, the transmitter 46 is replaced by a transceiver that is capable of transmitting and receiving signals. In the depicted embodiment, the transmitter 46 transmits in the ultra-high frequency (UHF) range but those of ordinary skill in the art would understand that other ranges may equally be used. Further, while in FIG. 3 the transmitter 46 is depicted as a radio chip, other methods and devices for sending a radio wave may be used.

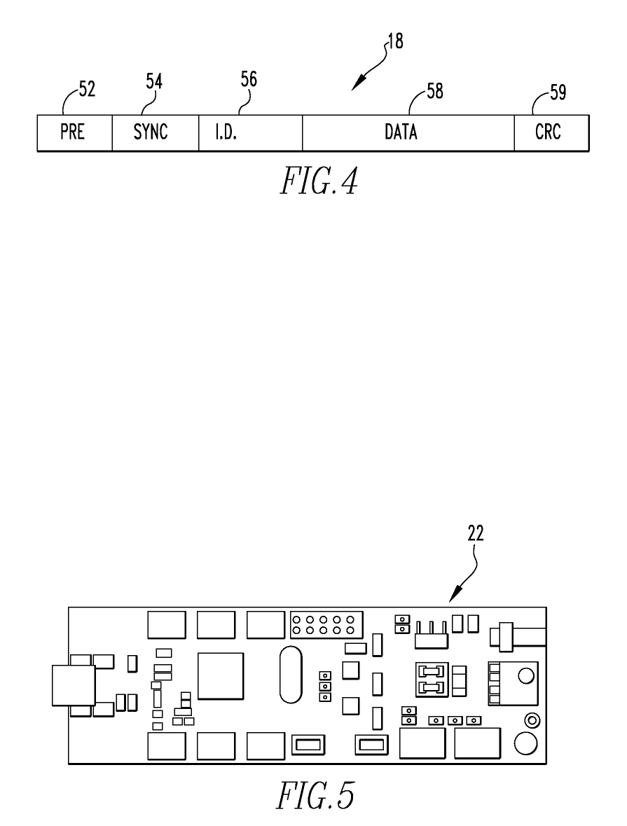

The transmitter 44 transmits data in the form of a packet. At a minimum, the packet includes control information and the actual data. FIG. 4 illustrates an exemplary digital data packet structure 18. The data packet structure 18 includes a pre-amble 52, a sync flag 54, an implant identifier 56, data 58, and error checking data 59. The pre-amble 52 initializes the receiver, and the sync flag 54 detects the incoming packet. The telemetry data 58 may be any physical measurement, such as implant forces, implant micro-motion, implant position, alkalinity, temperature, pressure, etc. The error checking data 59 is used to verify the accuracy of the data packet. For example, the error checking data 59 may contain a value to calculate a checksum or cyclic redundancy check. If the data is corrupted, it may be discarded or repaired. In some embodiments, the data packet 18 also may include a length field that provides data as to the length of the packet. For example, if the implant has multiple sensors, then length field may indicate a larger data packet than if the implant has only a single sensor. In some embodiments, the data packet structure may include fields for encryption.

FIG. 5 illustrates an example of the receiver 22. In the depicted embodiment, the receiver 22 is a USB wireless adapter capable of receiving radio waves adapted for connection to the processor 20. For example, the USB wireless adapter may be a development board having a microcontroller with on-board flash memory and USB interface support to provide a flexible platform for software development, such as the AT90USB 1286 development board available from ATMEL Corporation, 2325 Orchard Parkway, San Jose, California 95131. The receiver 22 may include software such that it is recognized by the processor 20 as a USB mass storage device. The receiver 22 may be used to develop “Software Defined Radio” (SDR) demodulation. An SDR system is a radio communication system that can potentially tune to any frequency band and receive any modulation across a large frequency spectrum through the use of as little hardware as possible and processing the signals through software.

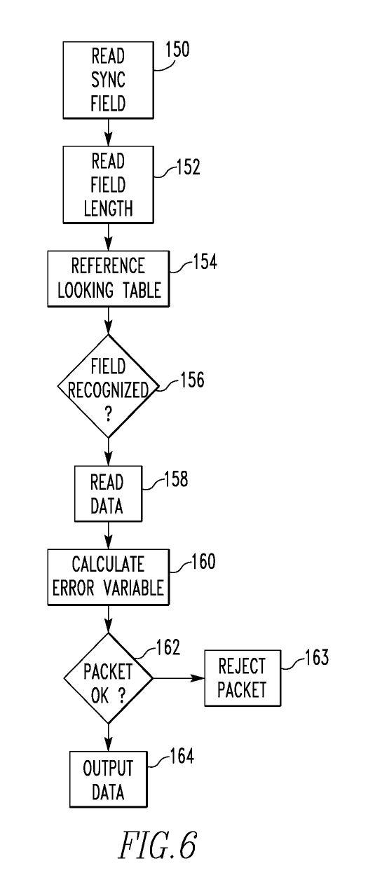

FIG. 6 illustrates an exemplary flowchart depicting the steps that may be taken by the receiver 22 upon receipt of the data packet structure 18 and initialization by the preamble field 52. In step 150, the receiver 22 recognizes the sync field 52. In optional step 152, the receiver 22 may read the length field. In step 154, the receiver 22 decodes the identification field 56. Step 154 may involve reference to a look-up table to match the identification field to a stored set of data. For example, the receiver may match the identification field with an entry in a database which contains information on the implant and/or the patient. Optional step 156 is decision whether or not the identification field is recognized. If the identification field is not recognized, the data packet may be rejected. Otherwise, the receiver proceeds to step 158. In step 158, the data 58 is read. In step 160, the error checking data 59 is calculated. In step 162, there is a decision as whether the data is error free. If the data packet contains an error, then the packet is rejected. Otherwise, the data is output to the processor 20, either through wire or wirelessly. As examples, the data may be output through a serial port or universal serial bus.

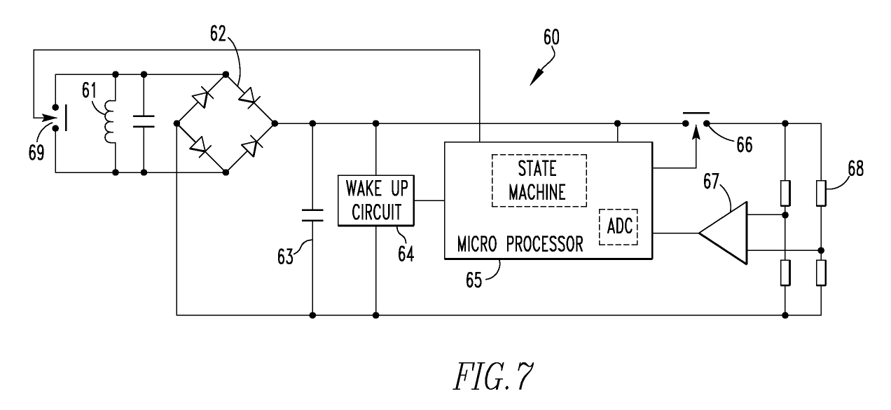

In some embodiments, the orthopaedic implant 12 includes on-board electronics for power harvesting, sensing data, processing of the sensed data, and data transmission. FIG. 7 illustrates an exemplary wiring diagram of a circuit 60. The circuit 60 includes an LC circuit 61, a bridge rectifier 62, a storage capacitor 63, a wake up circuit 64, a microprocessor 65, an enable measurement switch 66, an amplifier 67, a sensor and wheat stone bridge assembly 68, and a modulation switch 69. In the depicted embodiment, the wake up circuit 64 compares working voltage to stored voltage to see if the stored voltage reaches a certain threshold. As an example, the microprocessor 65 has a clock speed of 128 khz.

The LC circuit 61 receives a carrier signal from the antenna 14 to inductively power the on-board electronics. As an example, the carrier signal may have a frequency of about 125 kHz. The use of inductive power eliminates the requirement for a battery in the telemetric implant 12. In the depicted embodiment, the storage capacitor 63, a battery (not shown) or other energy storage device may be used to power the on-board electronics when not inductively powered. In other embodiments, the on-board electronics operate only when powered inductively from the antenna 14. The circuit 60 does not transmit raw data to the receiver 22 but instead modulates a load signal. This technique uses less power than raw transmission. The signal can be modulated using software embedded in the microprocessor 65. The load signal is related to the amount of resistance measured by the sensor assembly 68. In the depicted embodiment, the load signal is modulated at a frequency between 5 kHz and 6 kHz but those skilled in the art would understand that other frequency bands may be used. The change in load on the telemetric implant 12 is transmitted by the LC circuit 61 and received by the receiver 22.

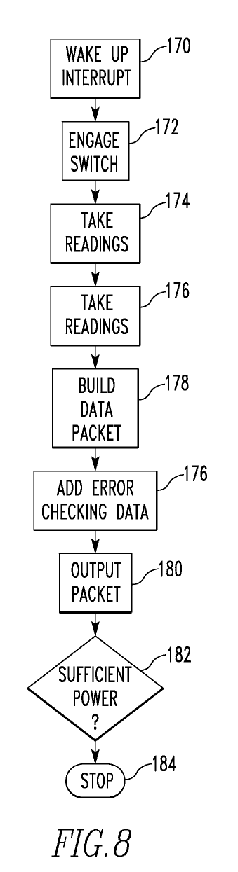

FIG. 8 is a flowchart that illustrates the steps taken within the circuit 60 for sensor measurement. In step 170, there is provided a wake-up interrupt by the wake up circuit 64. The wake up circuit 64 engages the enable measurement switch 66 in step 172 when the stored voltage reaches a certain threshold. This enables the sensor assembly 68 and powers the amplifier 67. The microprocessor 65 takes readings in step 174. The microprocessor 65 includes an analog-to-digital converter that converts the analog signal from the sensor assembly to a digital signal. In step 176, the microprocessor 65 forms a data packet, and generates an error checking data in step 178. In step 180, the microprocessor 65 outputs the data packet. In some embodiments, this may be accomplished by transmitting the data via a radio chip. In the embodiment depicted in FIG. 7, the microprocessor 65 selectively opens and closes the modulation switch 69 to send out the data via the LC circuit 61. In step 182, there is a decision whether there is sufficient power to resend the data packet. If so, the process loops back to step 180 to resend the data packet until all of the energy stored in the storage device 63 has been used. When there is no longer sufficient power to resend the data packet, the process stops in step 184. In the depicted embodiment, the wake up circuit 64 turns on above 3 volts and shuts down below 2 volts.

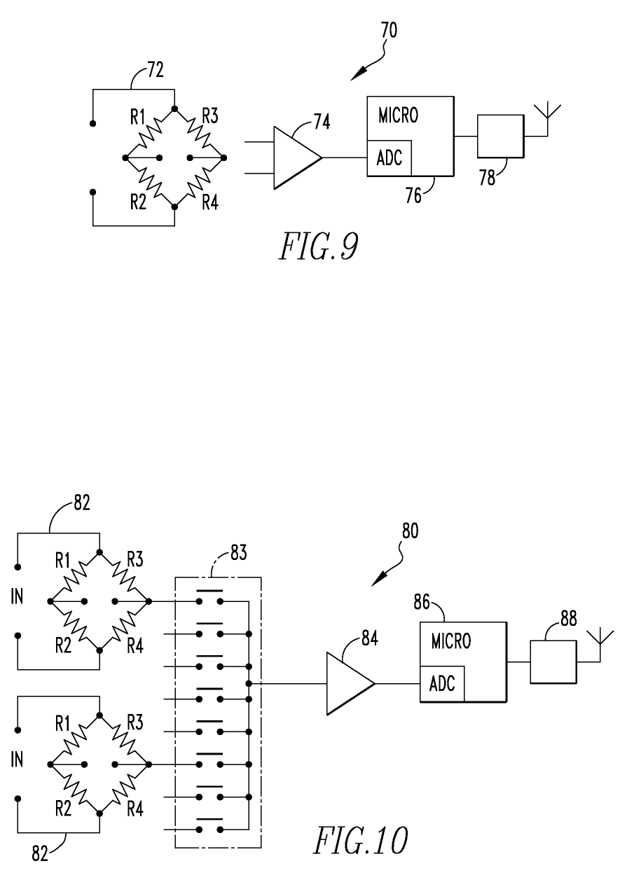

FIG. 9 schematically illustrates a first embodiment of on-board implant electronics 70. In FIG. 9, some components, such as a power supply, have been removed for clarity. The on-board implant electronics 70 includes a sensor and wheatstone bridge assembly 72, an amplifier 74, a microprocessor 76, and a transmitter 78. In the depicted embodiment, the sensor assembly 72 includes a foil gauge connected to a wheatstone bridge. Alternatively, the sensor may be a semiconductor or thin film strain gauge. The sensor assembly 72 may include any number of types of sensors including, but not limited to, a foil strain gauge, a semi-conductor strain gauge, a vibrating beam sensor, a force sensor, a piezoelectric element, a fibre Bragg grating, a gyrocompass, or a giant magneto-impedance (GMI) sensor. Further, the sensor may indicate any kind of condition including, but not limited to, strain, pH, temperature, pressure, displacement, flow, acceleration, direction, acoustic emissions, voltage, electrical impedance, pulse, biomarker indications, such as a specific protein indications, chemical presence, such as by an oxygen detector, by an oxygen potential detector, or by a carbon dioxide detector, a metabolic activity, or biologic indications to indicate the presence of white blood cells, red blood cell, platelets, growth factors, or collagens. Finally, the sensor may be an image capturing device. The microprocessor 76 includes an analog-to-digital converter that converts the analog signal from the sensor assembly to a digital signal. When the sensor assembly 72 is powered, the sensor assembly 72 sends a signal to the amplifier 74, which amplifies the signal. The amplified signal is sent to the microprocessor 76, which converts the signal from analog to digital. The microprocessor forms a data packet from the digital signal and transmits the data packet via the transmitter 78.

FIG. 10 schematically illustrates a second embodiment of on-board implant electronics 80. In FIG. 10, some components, such as a power supply, have been removed for clarity. The on-board implant electronics 80 includes a plurality of sensor and wheatstone bridge assemblies 82, a multiplexer 83, an amplifier 84, a microprocessor 86, and a transmitter 88. In its simplest form, the multiplexer 83 is an addressable switch. The multiplexer 83 is linked to the microprocessor and selects the sensor from which to receive data. In the depicted embodiment, the sensor assembly 82 includes a foil gauge connected to a wheatstone bridge. Alternatively, the sensor may be a semiconductor strain gauge. The microprocessor 86 includes an analog-to-digital converter that converts the analog signal from the sensor assembly to a digital signal. When the sensor assemblies 82 are powered, each sensor assembly 82 sends a signal to the multiplexer 83. The multiplexer 83 sends the multiplexed signal to the amplifier 84, which amplifies the signal. The amplified signal is sent to the microprocessor 86, which converts the signal from analog to digital. The microprocessor forms a data packet from the digital signal and transmits the data packet via the transmitter 88. While only two sensor assemblies are shown in FIG. 10, those having ordinary skill in the art would understand that the implant 12 may have more than two sensor assemblies and may be limited only by the size and shape of the implant. Further, the configuration of the sensors also may be tailored to meet the requirements of the patient's fracture.

FIGS. 11-14 illustrate one particular embodiment of the orthopaedic implant 12. In the depicted embodiment, the orthopaedic implant 12 is an intramedullary nail but other implant types may be used. The orthopaedic implant 12 may include one or more cavities to receive on-board electronics. Alternatively, the cavities may be termed “pockets.” In the embodiment depicted in FIG. 11, the orthopaedic implant 12 includes a first cavity 90 and a second cavity 92. While in the depicted embodiment the first cavity 90 is generally orthogonal to the second cavity 92, those having ordinary skill in the art would understand that other arrangements are possible. For example, the first cavity 90 may be diametrically opposed to the second cavity 92. The first cavity 90 is adapted to receive on-board electronics 100, and the second cavity 92 is adapted to receive an antenna 110. Of course, these component locations may be reversed. Further, both components may be located within a single cavity in some embodiments. In some embodiments, the cavity may be tapered to match the overall shape of the implant. The use of multiple cavities allows for different methods of encapsulation for each cavity. Different methods of encapsulation may be required depending upon the materials used.

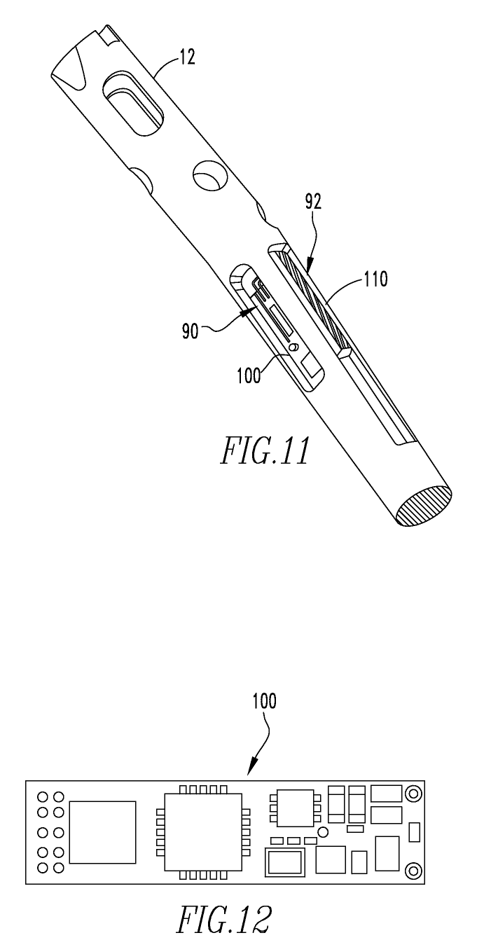

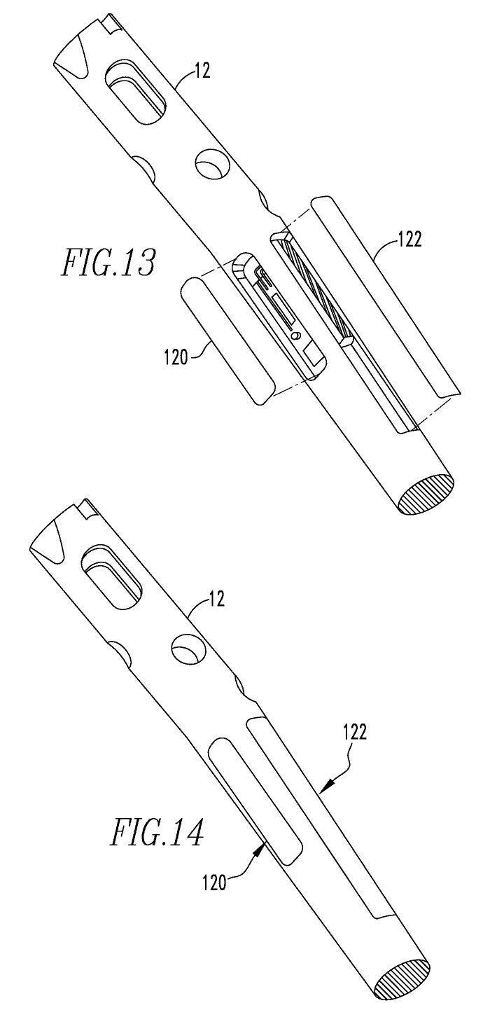

FIG. 12 illustrates an exemplary embodiment of the on-board electronics 100. The orthopaedic implant 12 may include one or more covers corresponding to the one or more cavities. In the embodiment depicted in FIGS. 13 and 14, there is provided a first cover 120 corresponding to the first cavity 90 and a second cover 122 corresponding to the second cavity 92. The one or more cavities may include a steeped recess to receive the cover. The cover is made from a biocompatible material. As examples, the cover may be made from titanium, stainless steel, shape memory alloy, or ceramic. Ceramics may include alumina, zirconia, boron nitride, or machinable aluminium nitride. In the embodiment depicted in FIGS. 13 and 14, the covers 120, 122 have a thickness in the range from about 43 microns to about 0.5 millimeters but of course other dimensions may be used. In some embodiments, a metal cover may affect the performance of the antenna, and therefore the electronics cavity may have a metal cover while the antenna has a ceramic cover. In some embodiments, the cover may include a ceramic central portion vapor deposited on a flange frame made of metal, such as titanium. In other embodiments, the cover may include a central foil portion and a metal flange frame to reduce the risk of signal loss.

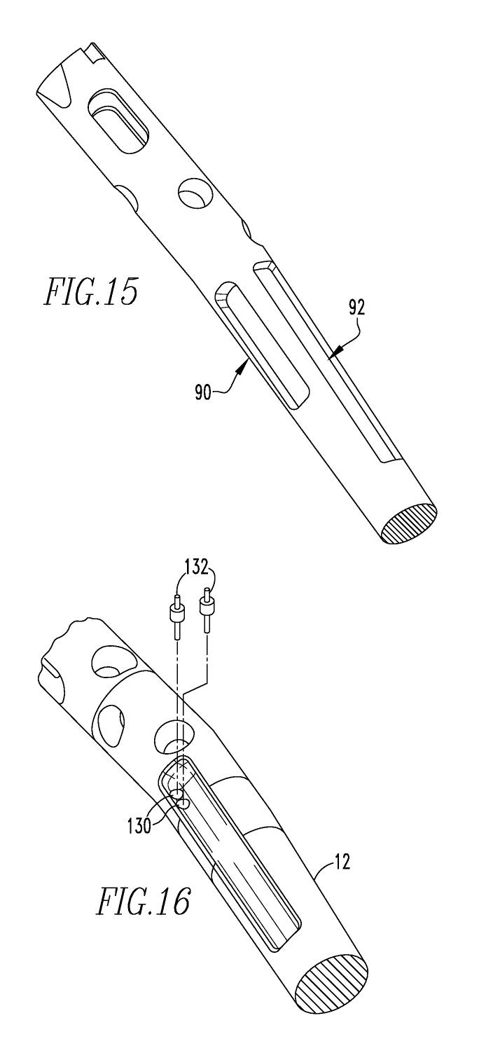

Consideration may be given to the location and size of the one or more cavities. The cavities should be conveniently placed but not significantly affect the structural integrity of the orthopaedic implant 12. Finite element analysis may be of use in judging appropriate cavity location and dimensions. Factors which may be considered include: (1) geometry of the implant; (2) symmetry of the implant (e.g., left and right implants); (3) whether the cavity provides a convenient location for data transmission and/or reception; (4) whether a sensor will be located in the same cavity as the embedded antenna coil; and (5) location of the largest bending moment applied to the implant. These factors are not all inclusive, and other factors may be of significance. Similar factors may be used to judge the dimensions of the one or more cavities. In the embodiment depicted in FIG. 15, the first cavity 90 is about 20 millimeters in length, about 5 millimeters in width, and about 3 millimeters in depth, and the second cavity 92 is about 30 millimeters in length, about 5 millimeters in width, and about 3 millimeters in depth. Other dimensions, however, may be equally used.

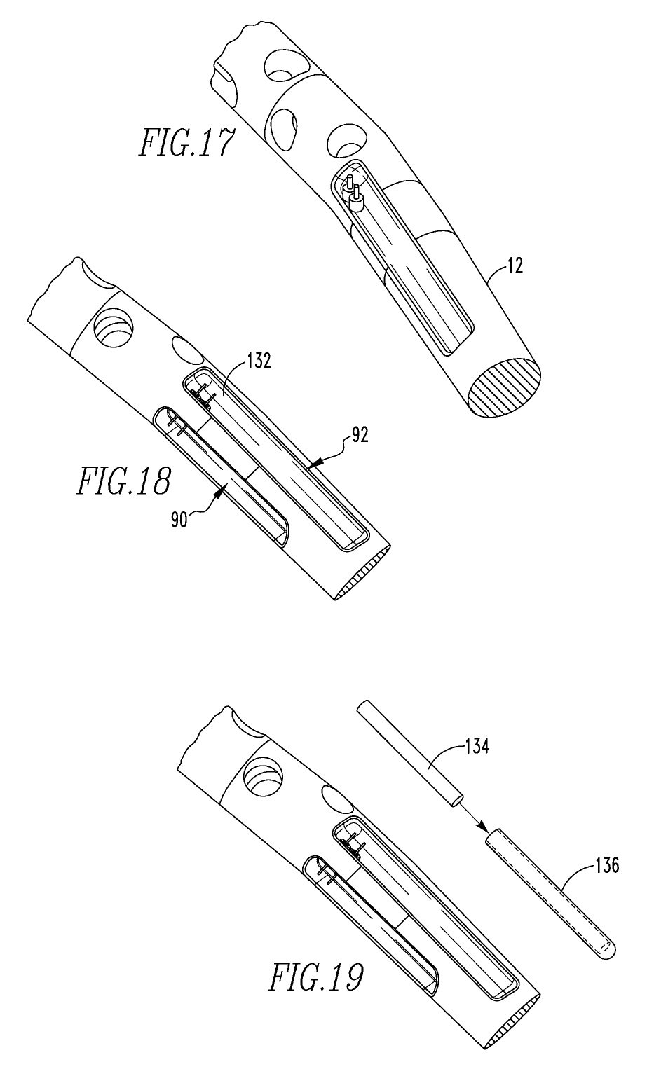

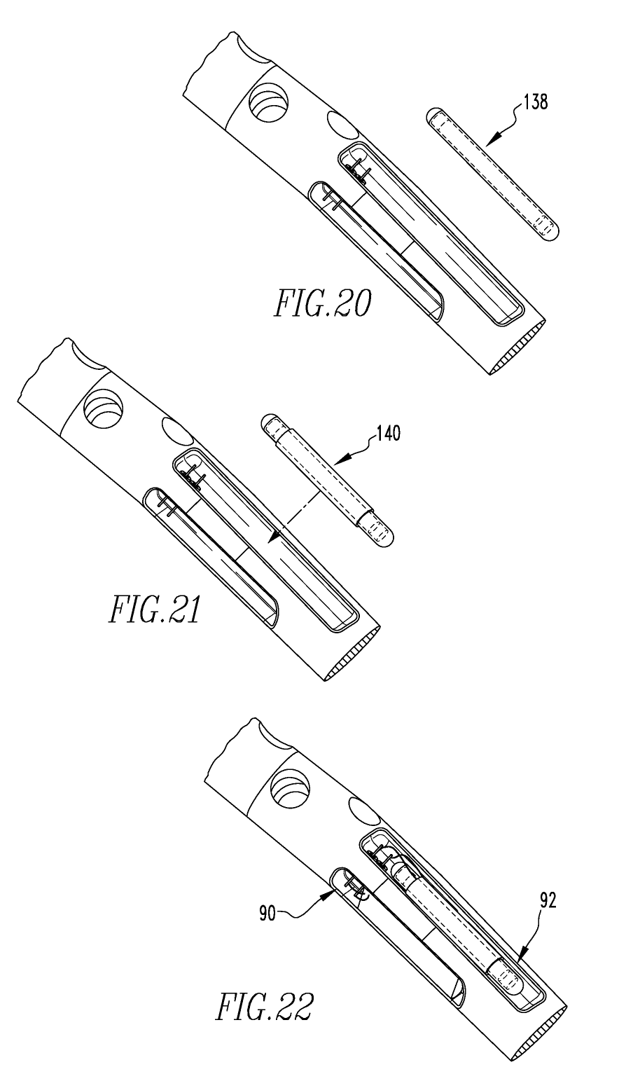

FIGS. 16-23 illustrate assembly of the orthopaedic implant 12 shown in FIGS. 11-14. As best seen in FIG. 16, one or more connection apertures 130 are placed in the implant 12 to connect the first cavity 90 to the second cavity 92. In some embodiments, the connection apertures 130 may be used to backfill the second cavity 92 with a polymer encapsulant (such as an epoxy or silicone elastomer) after attachment of the cover. Connectors 132 are placed in the holes 130 and may be affixed to the implant 12. For example, the connectors may be gold-brazed or laser welded to the implant. The implant 12 includes the biocompatible antenna 110. The antenna 110 includes a core 138 and wire 140 wrapped about the core. The core 138, which may be cylindrical or square-shaped in cross-section, includes a magnetically permeable material, such as ferrite. In FIG. 19, the core 138 is formed by a ferrite rod 134 placed within a borosilicate glass tube 136 but other materials or biocompatible coatings may be used. For example, the ferrite rod may be coated with a polyxylylene polymer, such as Parylene C. The glass tube 136 is sealed to contain the ferrite to make the core substantially biocompatible. For example, the glass tube may be sealed using an infrared laser. In some embodiments, the ferrite rod and/or the glass tube may be processed to include substantially planar portions for a better fit within the cavity. The core 138 is wrapped with wire 140, such as copper wire or gold plated steel wire. In the embodiment depicted in FIG. 21, there is about 300 turns of wire wrapped about the core 138. In an alternative embodiment, the wire 140 is wrapped about a ferrite rod and sealed within a glass tube while still allowing for external connection of the wire.

In addition or in the alternative, the on-board electronics and/or the antenna may be sealed by: (1) a compressed/deformed gold gasket to produce a hermetic seal; (2) electroplating over an epoxy capsule to produce a hermetic seal; (3) welding a ceramic lid with a metalized perimeter over the pick-up recess; or (4) coating the ferrite using a vapor-deposited material/ceramic.

As best seen in FIG. 22, the on-board electronics 100 is placed in the first cavity 90, and the antenna 110 is placed in the second cavity 92. In some embodiments, a sensor is placed under the on-board electronics 100. The on-board electronics 100 is electrically connected to the antenna 110 via the connectors 132. The on-board electronics 100 and/or the antenna 110 may be fixed in the cavities 90, 92 using a range of high stiffness adhesives or polymers including silicone elastomers, epoxy resins, polyurethanes, polymethyl methacrylate, ultra high density polyethylene terephthalate, polyetheretherketone, UV curable adhesives, and medical grade cyanoacrylates. As an example, EPO-TEK 301 available from Epoxy Technology, 14 Fortune Drive, Billerica, Massachusetts 01821. These types of fixation methods do not adversely affect the performance of the electrical components. In some embodiments, the cavities may include under cuts or a dovetail groove to hold the adhesive or polymer in place. Thereafter, the covers 120, 122 are placed on the implant 12 and welded in-place. For example, the covers may be laser welded to the implant.

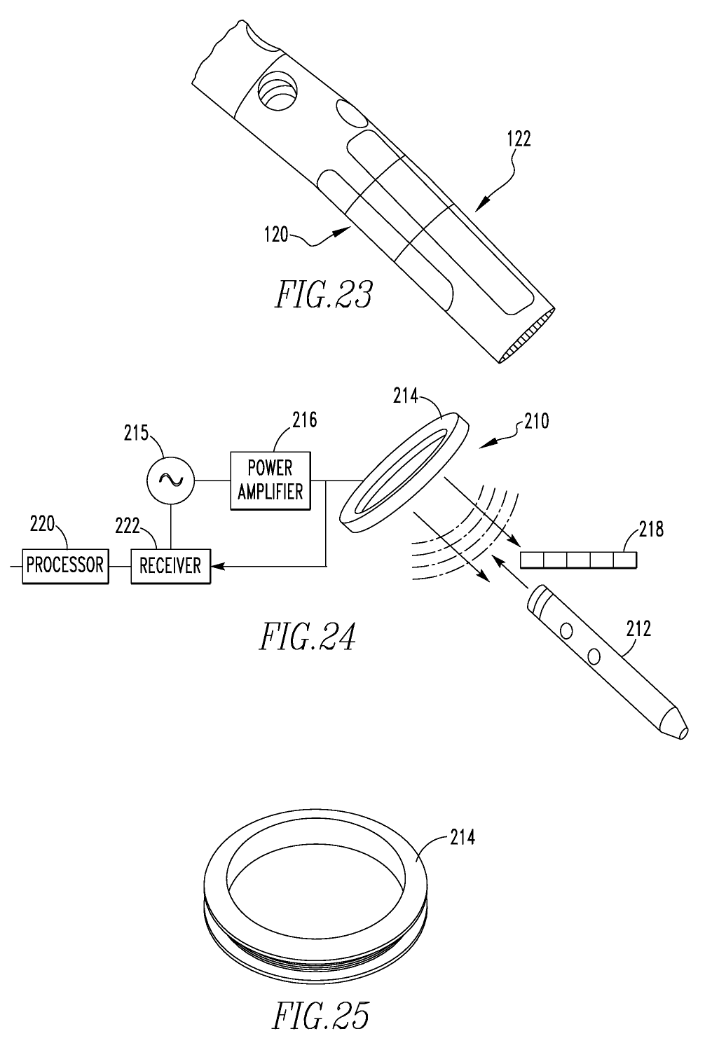

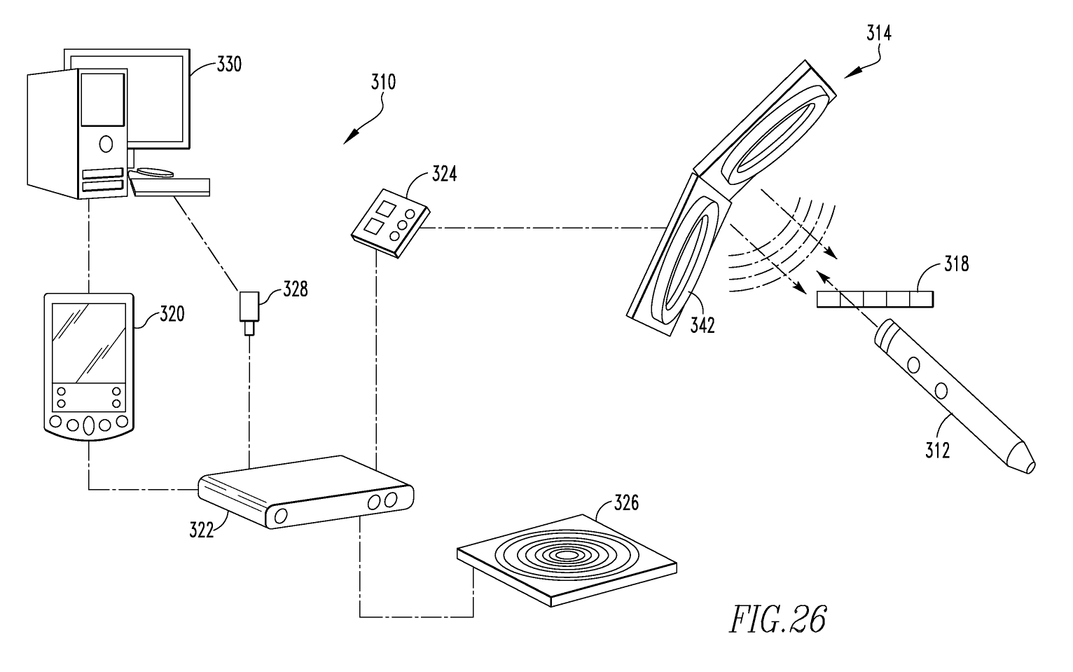

FIG. 24 illustrates a system 210 for communicating with an implant in a second embodiment. The system 210 includes an orthopaedic implant 212, a coil 214, a signal generator 215, an amplifier 216, a data packet 218, a processor 220, and a receiver 222. In the depicted embodiment, the orthopaedic implant 212 is an intramedullary nail but other types of orthopaedic implants may equally be used. As examples, the orthopaedic implant 212 may be an intramedullary nail, a bone plate, a hip prosthetic, or a knee prosthetic. Further, the processor 220 may be a desktop computer, a laptop computer, a personal data assistant (PDA), mobile handheld device, or a dedicated device. In some embodiments, the processor 220 and the receiver 222 form a single component. In the depicted embodiment, however, the receiver 222 is electrically connected to the processor 220 but is a separate component. The system 210 is similar to system 10 except that instead of the data packet being received by an antenna on the receiver 22, the data packet is received by the transmission coil 214 and sent by wire to the receiver 222. Alternatively, the coil 214 may be wirelessly connected to the receiver 222. Further, the coil 214, the amplifier 216, and/or the signal generator 215 may form a single component.

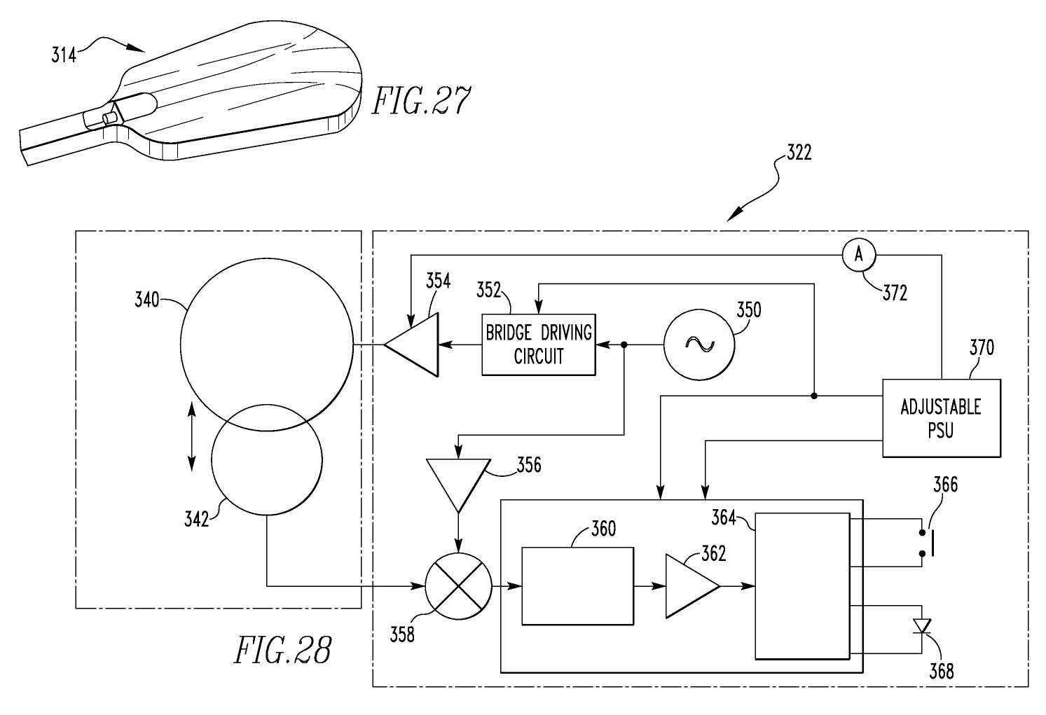

FIG. 25 illustrates the coil 214. In FIG. 25, the coil 214 is formed by a plastic spool wound with conductive wire. In the depicted embodiment, at least 60 turns of copper wire having a diameter of about 0.4 mm is wound onto the plastic spool, and the plastic spool has an inner diameter of about 100 mm, an outer diameter of about 140 mm, and a thickness of about 8 mm thickness using a semi-automated coil winding machine. However, these dimensions are merely exemplary and those having ordinary skill in the art would understand that other dimensions might be used.

FIG. 26 illustrates a system 310 for communicating with an implant in a third embodiment. The system 310 includes an orthopaedic implant 312, a paddle 314, a data packet 318, a first processor 320, and a control unit 322. In the depicted embodiment, the orthopaedic implant 312 is an intramedullary nail but other types of orthopaedic implants may equally be used. As examples, the orthopaedic implant 312 may be an intramedullary nail, a bone plate, a hip prosthetic, or a knee prosthetic. Further, the first processor 320 may be a desktop computer, a laptop computer, a personal data assistant (PDA), mobile handheld device, or a dedicated device. In some embodiments, the first processor 320 and the control unit 322 form a single component. In the depicted embodiment, however, the control unit 322 is electrically connected to the processor 320 but is a separate component. Optionally, the system 310 also may include a feedback indicator 324, a load scale 326, a portable storage device 328, and/or a second processor 330. The load scale 326 provides a reference for comparison. For example, in the case of an intramedullary nail, the load scale 326 may be used to compare the load applied to the patient's limb in comparison to the load placed on the intramedullary nail. As an example, the portable storage device 328 may be a flash memory device and may be integrated with a universal serial bus (USB) connector. The portable storage device 328 may be used to transfer data from the control unit 322 to a processor or from one processor to another. Moreover, the control unit 322 may be networked or incorporate a wireless personal network protocol.

The control unit 322 transmits a signal, the orthopaedic implant 12 receives the signal and transmits a data packet 318 containing data, the receiver 322 receives the data packet, and the processor 320 may either process the data or send the data to a storage device (not shown). As an example, the transmitted signal may be in the range from about 100 kHz to about 135 kHz.

The control unit 322 may transmit information by wire or wirelessly. The control unit 322 may use available technologies, such as ZIGBEE, BLUETOOTH, Matrix technology developed by The Technology Partnership Plc. (TTP), or other Radio Frequency (RF) technology. ZigBee is a published specification set of high level communication protocols designed for wireless personal area networks (WPANs). The ZIGBEE trademark is owned by ZigBee Alliance Corp., 2400 Camino Ramon, Suite 375, San Ramon, Calif., U.S.A. 94583. Bluetooth is a technical industry standard that facilitates short range communication between wireless devices. The BLUETOOTH trademark is owned by Bluetooth Sig, Inc., 500 108th Avenue NE, Suite 250, Bellevue Wash., U.S.A. 98004. RF is a wireless communication technology using electromagnetic waves to transmit and receive data using a signal above approximately 0.1 MHz in frequency. Due to size and power consumption constraints, the control unit 322 may utilize the Medical Implantable Communications Service (MICS) in order to meet certain international standards for communication. MICS is an ultra-low power, mobile radio service for transmitting data in support of diagnostic or therapeutic functions associated with implanted medical devices. The MICS permits individuals and medical practitioners to utilize ultra-low power medical implant devices, without causing interference to other users of the electromagnetic radio spectrum.

The feedback indicator 324 may include an audible and/or visual feedback system that informs the user when the implant is engaged and reliable data is being acquired. The feedback indicator 324 may be equipped with one or more signal “OK” light emitting diodes (LEDs) to provide feedback to the user on optimizing the position of the reader relative to the implant 12. In an exemplary case, the signal “OK” LED is illuminated when the signal frequency is between 5.3 kHz and 6.3 kHz and the signal is adequately received.

The paddle 314 includes a plurality of coils. In the embodiment depicted in FIG. 26, the paddle 314 includes a first coil 340 and a second coil 342, and the coils 340, 342 are angularly adjustable relative to another.

FIG. 27 illustrates an enclosure for the paddle 314. In the embodiment depicted in FIG. 27, there are two coils (not shown) that are generally parallel to another. The paddle 314 is used to provide power and telemeter data from the implant. In one particular embodiment, the coils are tuned to series resonance at about 125 kHz. In some embodiments, a drive frequency of 13.56 MHz may be selected because it is known to be a cleaner portion of the spectrum with less interference. The coils may be mechanically adjustable such that the coil centers may be moved toward or away from one another for nulling. Alternatively, AC coupling of the receiver coil reduces the magnitude of the RF carrier signal. The paddle 314 may be equipped with one or more LEDs and data capture buttons to enable measurements to be acquired by the user. The paddle 314 may include a wireless interface for connection to either a PDA or a PC. In some embodiments, the paddle 314 may be connected to the main power supply or battery powered for increased portability. The paddle 314 may include flexible coil bobbins to allow investigation of different coil formats (e.g. bifilar helical copper windings).

FIG. 28 illustrates a wiring diagram of the paddle 314 and the receiver 322. The paddle 314 includes a first coil 340 and a second coil 342. In the depicted embodiment, the first coil 340 is a transmission coil and the second coil 342 is a receiving coil but these functions may be reversed. The receiver 322 includes a signal generator 350, a bridge driving circuit 352, a coil driver 354, a buffer 356, a mixer 358, a band pass filter 360, a limiter 362, and an adjustable power supply unit 370. The receiver 322 also may include a processor 364, a switch 366, one or more light emitting diodes (LEDs) 368, and an ammeter 372. In the depicted embodiment, the band pass filter 360 generates a square wave, the mixing process is optimized for noise removal, the buffer 356 acts as a one-way gate to prevent interference, and the limiter 362 cleans the signal for conversion. In the depicted embodiment, data is incorporated into the backscatter of the carrier signal, and a “1” is indicated by 135.6 kHz and a “0” is indicated by 141 kHz. The power supply 370 is adjustable in the depicted embodiment, but may be non-adjustable in other embodiments. In the depicted embodiment, the receiver 322 operates for a period of time, such as 30 seconds, upon pressing the switch 366.

In some embodiments, the coil drive frequency may be automatically tuned to compensate for drift in resonant frequency of the reader coil and capacitors. Additionally, carrier cancellation may be achieved using digital signal processing (DSP) techniques to avoid the end-user manually tuning the coil. DSP techniques are also available to improve front-end filtering and reject out bands of interference.

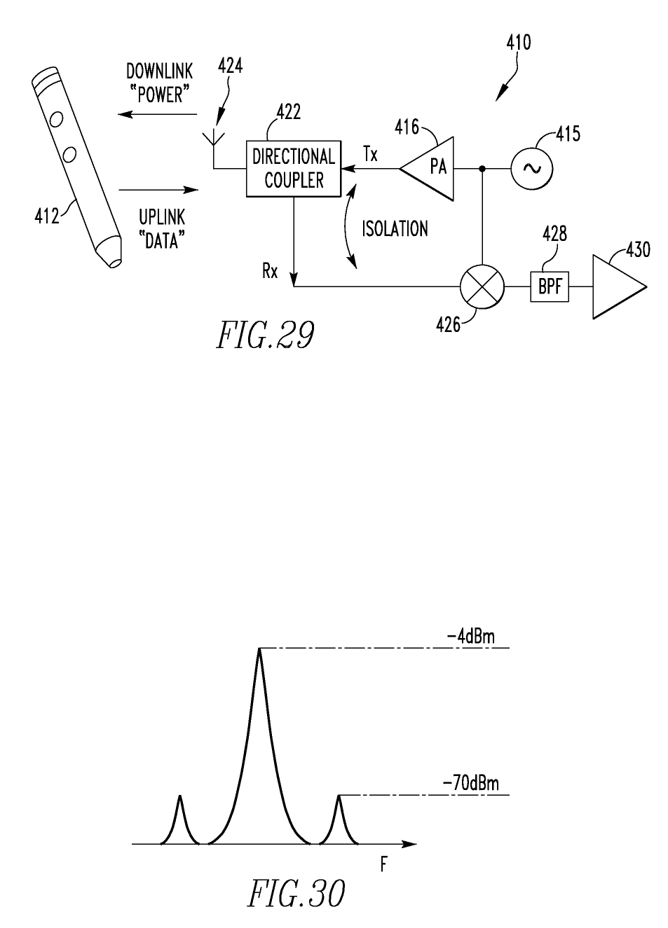

FIG. 29 illustrates a system 410 for communicating with an implant in a fourth embodiment. The system 410 includes an orthopaedic implant 412, a signal generator 415, a first amplifier 416, a directional coupler 422, an antenna 424, a mixer 426, band pass filter 428, and a second amplifier 430. The signal generator 415 generates a signal. The first amplifier 416 amplifies the signal. The directional coupler 422 allows the amplified signal to proceed through the antenna 424. The implant 412 receives the signal, takes a sensor measurement, and sends back a signal to the antenna 424. The directional coupler 422 routes the received signal to the mixer 426. The mixer 426 down shifts the frequency of the received signal. The band pass filter 428 strips out the desired the portion of the signal, and the second amplifier 430 amplifies the desired portion captured by the band pass filter. In some embodiments, the band pass filter is used to generate a square wave. Thereafter, the signal may be sent to another component for processing.

The system 410 utilizes homodyne detection. Homodyne detection is a method of detecting frequency-modulated radiation by non-linear mixing with radiation of a reference frequency, the same principle as for heterodyne detection. Homodyne signifies that the reference radiation (the local oscillator) is derived from the same source as the signal before the modulating process. The signal is split such that one part is the local oscillator and the other is sent to the system to be probed. The scattered energy is then mixed with the local oscillator on the detector. This arrangement has the advantage of being insensitive to fluctuations in the frequency. Usually the scattered energy will be weak, in which case the nearly steady component of the detector output is a good measure of the instantaneous local oscillator intensity and therefore can be used to compensate for any fluctuations in the intensity. Sometimes the local oscillator is frequency-shifted to allow easier signal processing or to improve the resolution of low-frequency features. The distinction is not the source of the local oscillator, but the frequency used.

FIG. 30 illustrates the signal after it is received and routed by the directional coupler 422. The band pass filter 428 is used to capture generally the wanted portions of the received signal.

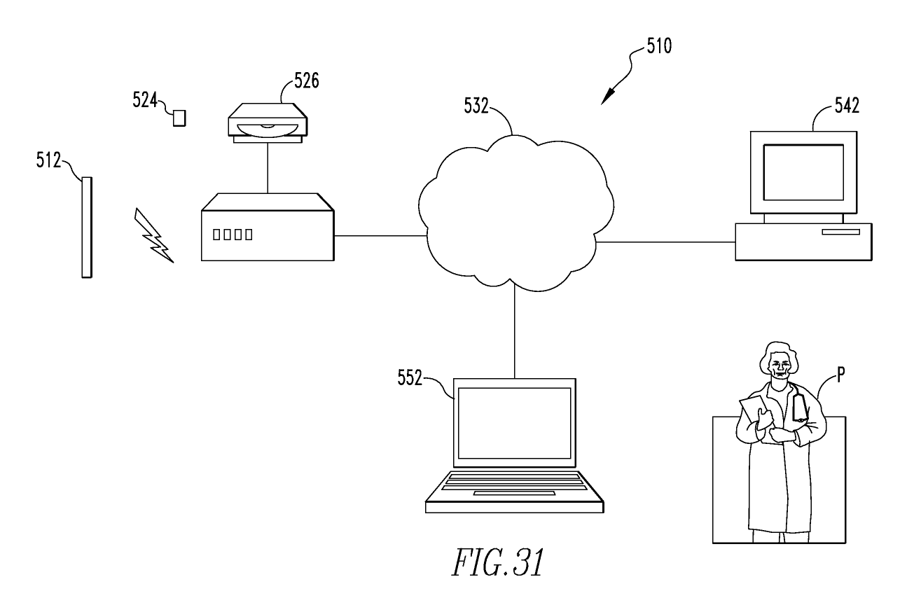

FIG. 31 illustrates a data storage system 510. The data storage system 510 includes an orthopaedic implant 512, a control unit 522, a network 532, a server 542, and a remote processor 552. Optionally, the data storage system 510 may include a portable storage device 524 and/or a peripheral storage device 526. Data is collected by the implant 512 and transmitted to the control unit 522. The data may be captured using an approved medical standard with rigorous protection and error checking of the data files. The data may be transferred to the portable storage device 524, the peripheral storage device 526, and/or the network 532. For example, the data may be sent to the server 542 via the network 532. As examples, the peripheral storage device 532 may be a hard disk drive or a media writer. A health care provider P may use the remote processor 552 to access and analyze the data from the implant 12. In one method, the health care provider P connects the portable storage device 524 to the remote processor and retrieves the data for analysis. In another method, the data is written to media using the peripheral storage device 526, and the health care provider P accesses data on the media using the remote processor. In yet another method, the health care provider P uses the remote processor to access the server via the network to retrieve stored implant data.

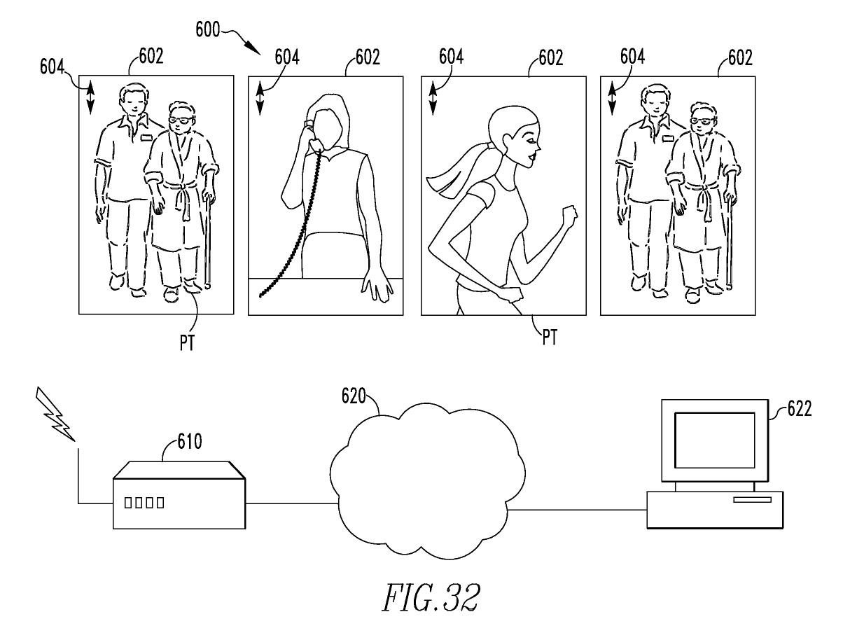

FIG. 32 illustrates a health care facility 600. The health care facility 600 includes one or more kiosks 602 and a receiver 610. Optionally, the health care facility 600 also may include a network 620 and/or a remote processor 622. The remote processor 622 may include internal or external devices for data storage. A patient PT having an implant 12, 212, 312, 412 enters the kiosk 602. The receiver 610 sends out a signal, the implant takes a sensor measurement, and sends the sensor data to the receiver. In some embodiments, the kiosk 602 further includes a relay 604. The relay 604 relays signals between the implant and the receiver. The receiver receives the one or more signals. In some embodiments, the receiver may process the received data and send the processed information to a healthcare provider. Alternatively, the receiver may send the data to the remote processor 622 via the network for remote processing and/or storage. In some embodiments, each kiosk 602 may have a weight sensor (not shown) to measure a load placed on the limb having the implant. In other embodiments, each kiosk 602 may have a visual protocol (not shown) of movements for the patient to execute while sensor measurements are taken. As examples, the visual protocol may be provided in the form of a static poster or electronic media.

As noted above, shielding the antenna may be necessary to allow for appropriate biocompatibility, but this often causes significant signal loss. One way to address the signal loss is to minimize the shielding (i.e, reduce the thickness of the cover) to allow for sufficient thickness for adequate biocompatibility while simultaneously minimizing the amount of signal loss. Another way to address this issue is to provide materials that minimize signal loss but allow for adequate biocompatibility. While non-metallics may be of interest, attaching a non-metallic cover to a metallic nail may provide manufacturing challenges. In yet another approach to address this issue, the antenna may be located in a cap attached to a portion of the implant. The cap may be non-mettalic, such as PEEK or ceramic, and an elastomeric seal, or the cap may be metallic with an epoxy sealant. For example, in the case of an intramedullary nail, the antenna may be located in a nail cap removably attached to the end portion of the nail In one other approach to address the issue of signal loss, the antenna may take the form of an umbilical cord which trails from the implant, as is commonly done in pacemakers and other implantable devices.

Although the depicted embodiments concentrate on the function of an instrumented intramedullary nail designed specifically for bone healing, alternative embodiments include incorporation of the sensor and other electronic components within other implantable trauma products, such as a plate, a bone screw, a cannulated screw, a pin, a rod, a staple and a cable. Further, the instrumentation described herein is extendable to joint replacement implants, such a total knee replacements (TKR) and total hip replacements (THR), dental implants, and craniomaxillofacial implants.

A patient receives a wireless instrumented joint reconstruction product. The electromechanical system within the implant may be used to monitor patient recovery using one or more sensors, and make a decision as to whether any intervention is required in the patient's rehabilitation. The telemetric joint replacement continuously measures a complete set of strain values generated in the implant and transmits them from the patient to a laboratory computer system without disturbing the primary function of the implant. Alternatively, a wired system may be utilized in the form of a wearable device external to the patient. Again, the electromechanical system could be designed to monitor various aspects of the patient's recovery.

The wireless technology may be introduced into dental implants to enable early detection of implant overloading. Overloading occurs when prolonged excessive occlusal forces applied to the implant exceeded the ability of the bone-implant interface to withstand and adapt to these forces, leading to fibrous replacement at the implant interface, termed “osseodisintegration,” and ultimately to implant failure. Again, a communication link may be used to selectively access the strain data in the memory from an external source.

The technology associated with the instrumentation procedure also may be adapted to monitor soft tissue repair (e.g. skin muscle, tendons, ligaments, cartilage etc.) and the repair and monitoring of internal organs (kidney's, liver, stomach, lungs, heart, etc.).

The advantage of the invention over the prior art concerns the incorporation of the components within the fixation device in a manner that protects the components, provides an accurate and stable connection between the sensor and its environment, maintains the functionality of the implant itself, and is suitable for large scale manufacture. The device allows for information to be gathered and processed yielding useful clinical data with respect to a patient's bone healing cascade.

The instrumented device removes the guessing from the conventional diagnostic techniques, such as x-ray, CT and MRI imaging, by providing the patient objective quantitative data collected from them through the healing process. Currently, there is no device which quantifies the skeletal loads encountered during fracture healing, as well as during different patient and physiotherapy activities. Furthermore, the load distribution between the implant and the adjacent bone during fracture healing is also unknown. Such data helps to optimize postoperative protocols for improved fracture healing and ultimately determine when the fixation device may be removed without the risk of re-fracture or causing too much pain to the patient.

In some embodiments, the signal generator generates a first signal, an amplifier amplifies the first signal, at least one coil transmits the amplified signal, an implant antenna receives the first signal and transmits a data packet containing data, a receiver receives the data packet, and a processor processes the data, sends the data to a data storage device, or retransmits the data to another processor. As an example, the step of processing the data may include the step of populating a database. As another example, the step of processing the data may include the step of comparing the data to a prior data packet or data stored in a database. In yet another example, the step of processing the data may include the step of statistically analyzing the data. In another example, the step of processing the data may include the steps of making a comparison to other data, making a decision based upon the comparison, and then taking some action based upon the decision. In yet another example, the step of processing the data may include the step of displaying the data, alone or in conjunction with other information, such as patient or statistical data.

In one particular embodiment, the step of processing the data may include the steps of comparing the data packet to statistical data stored in a database, deciding whether the data meets some minimum or maximum threshold, and taking appropriate action to achieve a healed state. In some embodiments, the step of processing the data may include iterating one or more steps until a desired outcome is achieved.

In one particular embodiment, the step of processing the data may include the steps of comparing the data packet to prior data stored in a database, determining a rate of change based upon the comparison. This further may include the step of comparing rates of change

In one particular embodiment, the step of processing the data may include the steps of comparing the data packet to statistical data stored in a database, deciding whether the data meets some minimum or maximum threshold, and outputting a recommended action to achieve a healed state. This may further include the step of automatically scheduling a revision surgery or identifying the next available time in the operating room for a revision surgery.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

1. A system for communicating patient information, the system comprising:
a medical implant, the medical implant having a first cavity and a second cavity, the first and second cavity connected by one or more apertures, the first cavity is adapted to receive on-board electronics, the on-board electronics comprising at least one sensor, a microprocessor, and a data transmitter, and the second cavity is adapted to receive an implant antenna;
a signal generator adapted to generate a first signal;
an amplifier electrically connected to the signal generator;
at least one coil electrically connected to the amplifier;
a receiver adapted to receive a data packet having data from the implant antenna; and
a processor connected to the receiver;
wherein the signal generator generates the first signal, the amplifier amplifies the first signal, the at least one coil transmits the amplified signal, the implant antenna receives the first signal and transmits a data packet containing data, the receiver receives the data packet, and the processor processes the data or sends the data to a data storage device.

2. The system of claim 1, wherein the processor is selected from the group consisting of a desktop computer, a laptop computer, a personal data assistant, a mobile handheld device, and a dedicated device.

3. The system of claim 1, wherein the receiver is an antenna with an adapter for connection to the processor.

4. The system of claim 1, wherein the on-board electronics comprise a plurality of sensor assemblies and a multiplexer.

5. (canceled)

6. The system of any of claim 1, wherein there are two coils, and the coils are housed within a paddle.

7. The system of claim 1, further comprising a control unit, and wherein the signal generator and the amplifier are housed within the control unit.

8-9. (canceled)

10. The system of claim 1, wherein the first cavity and the second cavity are orthogonal to one another.

11. The system of claim 1, wherein the first cavity and the second cavity are diametrically opposed.

12. The system of claim 1, wherein at least one of the first cavity and the second cavity further comprise a cover.

13. The system of claim 1, wherein the on-board electronics comprise an LC circuit, a bridge rectifier, a storage capacitor, a wake up circuit, a microprocessor, an enable measurement switch, an amplifier, a Wheatstone bridge assembly, and a modulation switch.

14. (canceled)

15. The system of claim 13, wherein the modulation switch modulates a load signal.

16. The system of claim 15, wherein the load signal is modulated at a frequency between 5 kHz and 6 kHz.

17. A method for communicating information from a medical device, the method comprising:
determining that a value of stored voltage in a communication system of a medical device exceeds a threshold wake-up voltage value;
enabling operation of a sensor device included in the medical device;
receiving a reading from the sensor device, the reading indicating one or more parameters associated with the medical device or a patient associated with the medical device;
forming a data packet including information representative of a received reading; and
wirelessly outputting the data packet.

18. The method of claim 17, further comprising determining that a value of stored voltage in the communications system no longer exceeds a threshold shut-down voltage and disabling one or more components of the communication system to prevent outputting data packets.

19. The method of claim 18, further comprising repeating receiving a reading, forming a data packet and wirelessly outputting the data packet until determining that the value of stored voltage no longer exceeds the threshold shut-down voltage.

20. The method of claim 17, further comprising receiving electrical energy from at least one of an energy scavenging device and an inductive coupling device, and storing the electrical energy in an electrical storage device of the communication system.

21. A medical implant, comprising:
a first cavity;
a second cavity, the second cavity connected by one or more apertures to the first cavity;
on-board electronics located in the first cavity, the on-board electronics comprising at least one sensor, a microprocessor, a data transmitter, an electrical energy storage device and a wake-up circuit; and
an antenna located in the second cavity and connected to the on-board electronics through the one or more apertures.

22. The implant of claim 21, further comprising an electrical energy scavenging device.

23. The implant of claim 21, further comprising a first cover, the first cover sealing the first cavity, and a second cover, the second cover sealing the second cavity, wherein the second cover includes a ceramic material.

24. The implant of claim 21, wherein the on-board electronics are configured to operate only when powered inductively from the antenna.
